(19) United States
(12) Patent Application Publication
(10) Pub. No.: US 2002/0151543 A1
(43) Pub. Date: Oct. 17, 2002

(54) Compositions and methods employing R (-) fluoxetine and other active ingredients

(75) Inventors: Timothy J. Barberich, Concord, MA (US); Paul D. Rubin, Sudbury, MA (US); Dean A. Handley, Westborough, MA (US)

Correspondence Address: PENNIE & EDMONDS LLP, 1667 K STREET NW, SUITE 1000, WASHINGTON, DC, 20006

(73) Assignee: Sepracor Inc., Marlborough, MA

(21) Appl. No.: 10/158,886

(22) Filed: Jun. 3, 2002

Related U.S. Application Data
(62) Division of application No. 09/664,732, filed on Sep. 19, 2000, now abandoned.
Application No. 09/664,732 is a continuation-in-part of application No. 09/177,703, filed on Oct. 23, 1998, now abandoned.
Application No. 09/177,703 is a continuation-in-part of application No. 09/086,262, filed on May 28, 1998, now abandoned.

(51) Int. Cl. A61K 31/5513; A61K 31/137

(52) U.S. Cl. 514/221; 514/651

(57) ABSTRACT

Pharmaceutical compositions which comprises R(−) fluoxetine and one or more other biologically active compounds are disclosed. Methods of treating or preventing a disease or disorder, especially a psychotic or psychiatric disease or disorder, using the above pharmaceutical composition or by administering a R(−) fluoxetine in combination with one or more other biologically active compounds are also disclosed. Methods of treating patients having or at risk of having AIDS or HIV infection, cancer, cardiac disorder, post-myocardial depression and posttraumatic stress disorder using optically pure R(−) fluoxetine in combination with one or more other biologically active compounds are further disclosed.

This application is a continuation-in-part of the U.S. patent application Ser. No. 09/086,262, filed May 28, 1998, the content of which is incorporated herein by reference.

### FIELD OF THE INVENTION

This invention relates to pharmaceutical compositions and methods of treatment or prevention employing two or more active ingredients.

### INTRODUCTION

The present invention encompasses pharmaceutical compositions which comprise, and methods which utilize, optically pure R(−) fluoxetine and a second biologically active compound, preferably, a benzodiazepine compound, a tricyclic antidepressant, a 5-HT_{1A} receptor antagonist, a 5-HT₃ receptor agonist, a β-adrenergic antagonist, an antipsychotic agent, an anti-anxiolytic or other psychotropic drug. The present invention is well suitable for treating patients, having AIDS or HIV infection, cancer, cardiac disorder, post-myocardial depression, posttraumatic stress disorder or psychiatric disorder.

### BACKGROUND OF THE INVENTION

Fluoxetine, N-methyl-3-phenyl-3-[(α,α,α-trifluoro-p-tolyl)-oxy]-propylamine, is marketed as the hydrochloride salt form of the racemic mixture under the tradename PROZAC ® (U.S. Pat. No. 4,314,081). PROZAC® is indicated for the treatment of depression, obsessive-compulsive disorder (OCD) and bulimia nervosa (Physician's Desk References® 52 Edition 1998, pages 859-860).

As disclosed in U.S. Pat. No. 5,708,035, the racemic mixture of fluoxetine has certain disadvantages including headaches, nervousness, anxiety, insomnia, akathisia, severe anxiety leading to intense violent suicidal thoughts and self mutilation, manic behavior, nausea, diarrhea, drowsiness, decrease in libido, and/or sexual dysfunction. The major disadvantage of the racemic mixture of fluoxetine is its long half-life and long duration of action. This long half life can lead to a buildup of fluoxetine in the patient's body and a concomitant increase in the above described side effects when a patient is given multiple doses. More importantly, its long half-life and long duration of action also makes it impossible or impractical to use the racemic mixture of fluoxetine as part of a combination therapy, although combination therapy might be very desirable.

R(−) fluoxetine, and its use, is described in U.S. Pat. Nos. 5,708,035 and 5,648,396 to Young et al. These patents state that the use of R(−) fluoxetine decreases the incidence of adverse effects seen with the racemic mixture of fluoxetine. These patents also state that R(−) fluoxetine is useful in the treatment of depression, migraine headaches, pain, in particular chronic pain, psychoactive substance abuse disorders and obsessive compulsive disorders. Additionally, U.S. Pat. No. 5,648,396 discloses a method of treating depression in a human by first administering R(−) fluoxetine followed by a subsequent administration of a monoamine oxidase inhibitor.

U.S. Pat. No. 5,356,934 discloses a method for treatins sleep apnea in mammals which comprises administering to mammal requiring sleep apnea treatment an effective amount of (R)-fluoxetine or a pharmaceutically acceptable acid addition salt or solvate thereof.

U.S. Pat. No. 4,035,511 discloses a method of producing analgesia or reducing hyperalgesia by administering racemic fluoxetine either alone or with morphine sulfate.

U.S. Pat. No. 4,329,356 discloses a method for lowering blood pressure in a hypertensive mammal which comprises the co-administration of racemic fluoxetine and 1-5-hydroxytryptophane, or preferably the co-administration of fluoxetine, 1-5-hydroxytryptophane and a peripheral decarboxylase inhibitor such as carbidopa[α-hydrazino-α-methyl-β-(3,4-dihydroxyphenyl) propionic acid monohydrate] or banserazide [n-(dl-seryl)-nu″-(2,3,4-trihydroxybenzyl)hydrazine].

U.S. Pat. No. 4,594,358 and U.S. Pat. No. 4,683,235 discloses, respectively, a method of potentiating dextropropoxyphene or codeine analgesia in mammals, either alone or in combination with aspirin or acetaminophen, which comprises the administration of an effective amount of racemic fluoxetine or norfluoxetine prior to, concomitantly with, or after the administration of dextropropoxyphene or codeine which, if given alone, would produce less than the desired analgesic effect.

U.S. Pat. No. 4,895,845 discloses a method of assisting weight loss in patients through the concomitant administration of a rauwolfia alkaloid and at least one antidepressant selected from the group consisting of aminoazales, phenoxyphenylpropylamine antidepressants such as fluoxetine, and aminopropiophenones, or preferably through the concomitant administration of a rauwolfia alkaloid, at least one antidepressant selected from the group consisting of aminoazales, phenoxyphenylpropylamines, and aminopropiophenones, and one or more of the sympathomimetic anorectic agents that have proved successful in some instances in promoting patient compliance to a calorically-restricted diet.

U.S. Pat. No. 5,512,593 discloses a method of treating depression comprising administering to a patient an opioid antagonist selected from the group consisting of naltrexone, naloxone and a compound selected from the group consisting of one or more non-tricyclic antidepressants exhibiting serotonin reuptake inhibition in the synapses of the central nervous system such as bupropion, sertraline, fluoxetine, and trazodone.

U.S. Pat. No. 5,527,788 discloses the use of DHEA and at least one anorectic agent, such as fenfluramine HCl, phentermine HCl, phendimetrazine tartrate, mazindol, diethylpropion HCl, and fluoxetine HCl, in combination to produce weight loss in animals.

U.S. Pat. No. 5,532,268, U.S. Pat. No. 5,538,992 and U.S. Pat. No. 5,552,429 discloses, respectively, a method for potentiating the action of fluoxetine, in increasing the availability of serotonin, norepinephrine and dopamine in the brain, comprising administering fluoxetine to a patient in need thereof in combination with a second component which is chosen from the group consisting of alprenolol, WAY 100135, spiperone, propranolol and tertatolol, pindolol, or a compound of the formula I that is described therein.

WO 92/00103 discloses a pharmaceutical product comprising two or three active ingredients selected from a 5-HT₃ receptor antagonist, a 5-HT re-uptake inhibitor such as fluoxetine and a 5-HT₁ receptor agonist, as a combined preparation for simultaneous, separate or sequential use in therapy of depression and/or migraine.

WO 96/09044 discloses compositions for the effective treatment of a variety of chronic and intractable disorders, such as intractable coughing, dermatitis, chronic pain and tinnitus, which failed to respond to other treatments. The compositions are a therapeutically effective dosage of dextromethorphan (DM) in combination with a therapeutically effective dosage of an inhibitor of enzymatic dextromethorphan oxidation by the liver enzyme debrisoquin hydroxylase. As disclosed therein, antioxidants include yohimbine, fluoxetine, haloperidol, ajmaline, lobeline, pipamperone.

WO 96/09047 discloses a method of treating alcoholism and alcohol dependence in a mammal comprising administering to the mammal a therapeutically effective amount of a synergistic combination of at least one opioid antagonist, and at least one selective serotonin reuptake inhibitor. The phrase “selective serotonin reuptake inhibitor”, as used therein, denotes compounds which enhance brain serotonergic activity by blocking the neuronal reuptake and subsequent inactivation of serotonin at synaptic junctions between nerve cells; such include fluoxetine, sertraline, paroxetine, venlafaxine, fluvoxamine, nefazodone, citalopram, and zimeldine.

WO 97/31629 discloses a method of treating disorders of sleep comprising administering to a patient in need of such treatment a first component chosen from the group consisting of fluoxetine, venlafaxin, citalopram, fluvoxamine, paroxetine, milnacipran and duloxetine in combination with a second component chosen from the group consisting of alprenolol, WAY 100135, WAY 100635, spiperone, pindolol, (S)-UH-301, penbutolol, propranolol, tertatolol, and a compound of the formula I as described therein.

From the above, it appears that the art has been exploring the use of racemic fluoxetine in combination with other drugs for a number of uses. However, there is a need for combination therapy with the advantages of racemic fluoxetine without the disadvantages of racemic fluoxetine such as the great potential for adverse drug-drug interactions.

### SUMMARY OF THE INVENTION

The present invention encompasses a pharmaceutical composition which comprises an effective amount of optically pure R(−) fluoxetine, or a pharmaceutically acceptable salt thereof, substantially free of its S(+) stereoisomer, an effective amount of second biologically active compound and a pharmaceutically acceptable carrier. The present invention also relates to a method of treating or preventing diseases or disorders in humans, especially a psychotic or psychiatric disease or disorder, using such pharmaceutical compositions.

The present invention also encompasses a method of treating or preventing a psychotic or psychiatric disease or disorder by administering optically pure R(−) fluoxetine and a second biologically active compound, preferably a benzodiazepine compound, a tricyclic antidepressant, an antipsychotic agent, an anti-anxiolytic, a β-adrenergic antagonist, a 5-HT_{1A} receptor antagonist, a5-HT₃ receptor agonist, or a compound selected from the group consisting of buspirone, hydroxyzine, lorazepam, olanzapine, quetiapine, risperidone, 9-hydroxy-risperidone, sertindole, tomoxetine, valium and ziprasidone. The present invention further encompasses methods of treating patients, having or at risk of having AIDS or HIV infection, cancer, cardiac disorder, post-myocardial depression, posttraumatic stress disorder or other psychiatric disorders, using optically pure R(−) fluoxetine in combination with one or more biologically active compounds.

Finally, the present invention encompasses methods of treating or preventing a psychotic or psychiatric disease or disorder which comprises administering an effective amount of R(−) fluoxetine and an amount of a biologically active5-HT_{1A} receptor antagonist for a first period of time, and after the first period of time, administering an effective amount of R(−) fluoxetine and a reduced amount of said biologically active 5-HT_{1A} receptor antagonist for a second period of time; and kits to be used for such treatment or prevention.

Thus, the invention encompasses the use of R(−) fluoxetine, salts, solvates, hydrates and pro-drugs thereof in combination with one or more additional active ingredients. R(−) fluoxetine can be used in the same pharmaceutical composition as the one or more additional active ingredient, or R(−) fluoxetine can be administered concurrently or sequentially with the other active ingredients, i.e., before, during or after.

The present invention does not contemplate a pharmaceutical composition containing an effective amount of optically pure R(−) fluoxetine and an effective amount of a monoamine oxidase inhibitor. The present invention also does not encompass methods of treatment or prevention using optically pure R(−) fluoxetine in combination with a monoamine oxidase inhibitor.

Specific preferred combinations and further details of the invention are described in detail below.

### THE DETAILED DESCRIPTION OF THE INVENTION

The present invention encompasses pharmaceutical compositions which comprise a therapeutically effective amount of R(−) fluoxetine or a pharmaceutically acceptable salt thereof, substantially free of its S(+) stereoisomer, an effective amount of second biologically active compound, preferably, a benzodiazepine compound, a tricyclic antidepressant, an antipsychotic agent, a5-HT_{1A} receptor antagonist such as pindolol, a 5-HT₃ receptor agonist, a β-adrenergic antagonist such as pindolol, an anti-anxiolytic, or a compound selected from the group consisting of buspirone, hydroxyzine, lorazepam, olanzapine, quetiapine, risperidone, 9-hydroxy-risperidone, sertindole, tomoxetine, valium and ziprasidone, and a pharmaceutically acceptable carrier.

Any pharmaceutically acceptable salt of R(−) fluoxetine can be used in the present invention; however, acid addition salts are preferred. The most preferred pharmaceutically acceptable salt of R(−) fluoxetine is the hydrochloride salt.

The compositions and methods of this invention employ the optically pure R(−) isomer of fluoxetine in combination with drugs that act on the central nervous system (“CNS”), such as 5-HT_{1A} receptor, including hypnotics and sedatives; drugs useful in treating psychiatric disorders, such drugs include antipsychotic or neuroleptic drugs, antianxiety drugs, antidepressants and mood-stabilizers; central nervous system stimulants such as amphetamines; dopamine receptor agonists; antimonic agents; antipanic agents; and antipsychotic agents and the like. R(−) fluoxetine may also be used in combination with cardiovascular agents (beta blockers and ACE inhibitors); antivirals; antibiotics, antifungals; antineoplastics; and others.

The present invention provides the skilled artisan with a substantial benefit, that is, the ability to use a SSRI compound, such as R(−) fluoxetine, with one or more other active drugs without, or with reduced concern for, adverse drug-drug interactions. For example, R(−) fluoxetine can be used in a number of different patient populations including but not limited to cancer patients, cardiac patients, AIDS patients or HIV infected populations, and psychiatric patients, each of which is likely undergoing treatment with a drug other than racemic fluoxetine or is enantiomers. Further, many patients affiliated with life threatening diseases experience depression particularly at or about the time they first learned of their affliction. For example, victims of heart attacks are known to experience depression. Now these patients can be treated for their cardiac disorder as well as be treated for their depression while avoiding the adverse effects associated with the administration of racemic fluoxetine.

Moreover, the use of R(−) fluoxetine in combination with the drugs described herein provides an improved therapy over certain therapies presently available in the art; the use of R(−) fluoxetine and certain of the drugs may allow for treatment where none was previously available; and certain combinations can provide a synergistic benefit.

In a preferred embodiment, R(−) fluoxetine is used in combination with a benzodiazepine compound, a tricyclic antidepressant, an antipsychotic agent, an anti-anxiolytic, a β-adrenergic antagonist, a 5-HT_{1A} receptor antagonist, a 5-HT₃ receptor agonist, or one or more of the following compounds: buspirone, hydroxyzine, lorazepam, olanzapine, quetiapine, risperidone, 9-hydroxy-risperidone, sertindole, tomoxetine, valium and ziprasidone.

The preferred benzodiazepine compounds for use within the present invention include, but are not limited to, alprazolam, brotizolam, chlordiazepoxide, clobazam, clonazepam, clorazepate, demoxepam, diazepam, estazolam, flumazenil, flurazepam, halazepam, lorazepam, midazolam, nitrazepam, nordazepam, oxazepam, prazepam, quazepaml temazepam or triazolam.

The preferred tricyclic antipsychotic compounds for use within the present invention include, but-are not limited to, chlorpromazine, mesoridazine, thioridazine, acetophenazine, fluphenazine, perphenazine, trifluoperazine, chlorprothixene, thiothixene, clozapine, haloperidol, loxapine, molindone, pimozide, risperidone, 9-hydroxy-risperidone or desipramine.

The preferred 5-HT_{1A} receptor antagonists for use within the present invention include, but are not limited to, alprenolol, WAY 100135, spiperone, pindolol, (S)-UH-301, penbutolol, propranolol, tertatolol, and a compound of the formula I as disclosed in U.S. Pat. No. 5,552,429, the content of which is herein incorporated by reference.

In a preferred embodiment, a pharmaceutical composition of the invention comprises from about 5 mg to about 100 mg of R(−) fluoxetine, and the second biologically active compound, which is a benzodiazepine compound, a tricyclic antidepressant, an antipsychotic agent, an anti-anxiolytic, a β-adrenergic antagonist, a 5-HT_{1A} receptor antagonist, a 5-HT₃ receptor agonist, or a compound selected from the group consisting of buspirone, hydroxyzine, lorazepam, olanzapine, quetiapine, risperidone, 9-hydroxy-risperidone, sertindole, tomoxetine, valium and ziprasidone. The dosage of the second biologically active compound can be easily determined by skilled artisans, e.g., following dosages recommended in the Physician's Desk Reference® 52 Edition 1998.

In a preferred pharmaceutical composition, the R(−) fluoxetine is present in an amount on weight basis, relative to S(+) fluoxetine, from about 100:1 to about 10,000:1.

The present invention also relates to a method of treating or preventing a disease or disorder, especially a psychotic or psychiatric disease or disorder, using the above pharmaceutical composition.

The present invention further encompasses methods of treating or preventing a disease or disorder by administering R(−) fluoxetine in combination with a second biologically active compound which is a benzodiazepine compound, a tricyclic antidepressant, an anti-anxiolytic, a β-adrenergic antagonist, a 5-HT_{1A} receptor antagonist, a 5-HT₃ receptor agonist, or a compound selected from the group consisting of buspirone, hydroxyzine, lorazepam, olanzapine, quetiapine, risperidone, 9-hydroxy-risperidone, sertindole, tomoxetine, valium and ziprasidone.

The diseases or disorders that can be treated or prevented using R(−) fluoxetine and a benzodiazepine include, but are not limited to, depression, anxiety, alcohol withdrawal, preoperative apprehension, Lennox-Gastaut syndrome (petit mal variant), bulimina nervosa, seizures, bipolar disorders, panic disorder, skeletal muscle spasm, spasticity caused by upper motor neuron disorders, athetosis and stiffman syndrome, convulsive disorders and insomnia.

The diseases or disorders that can be treated or prevented using R(−) fluoxetine and a tricyclic antipsychotic compound include, but are not limited to, anxiety, depression, nausea, vomiting, restlessness and apprehension before surgery, acute intermittent porphyria, tetanus, intractable hiccups, severe behavioral problems and hyperactivity in children, emotional withdrawal, conceptual disorganization, tension, hallucinatory behavior, suspiciousness and blunted affect in schizophrenic patients or Tourette's Disorder.

The diseases or disorders that can be treated or prevented using R(−) fluoxetine and a 5-HT_{1A} receptor antagonist include, but are not limited to, depression, obsessive-compulsive disorders, obesity and late luteal phase syndrome, hypertension, migraine, essential tremor, hypertrophic subaortic stenosis and pheochromocytoma. The more preferred disease or disorder that can be treated or prevented using R(−) fluoxetine and a 5-HT_{1A} receptor antagonist includes, but is not limited to, posttraumatic depression disorder.

The diseases or disorders that can be treated or prevented using R(−) fluoxetine and a compound selected from the group consisting of buspirone, hydroxyzine, lorazepam, olanzapine, quetiapine, risperidone, 9-hydroxy-risperidone, sertindole, tomoxetine, valium and ziprasidone include, but are not limited to, anxiety, Attention Deficit Disorders, depression, hypertension, schizophrenia and psychotic disorders.

The disease or disorder that can be treated or prevented using R(−) fluoxetine and a β-adrenergic antagonist includes, but is not limited to, post myocardial infarction depression. The preferred β-adrenergic antagonists include, but are not limited to, S(−) pindolol, penbutolol and propranolol.

As used in the present application, the terms “substantially free of the S(+) stereoisomer” and “optically pure R(−) fluoxetine” means that the composition contains at least 90% by weight of R(−) fluoxetine and 10% by weight or less of S(+) fluoxetine. In the most preferred embodiment the “substantially free of the S(+) stereoisomer” means that the composition contains at least 99% by weight R(−) fluoxetine and 1% or less of S(+) fluoxetine.

Optically pure R(−) fluoxetine can be asymmetrically synthesized or it can be resolved using conventional techniques, both of which are known in the art. For example, the synthesis of R(−) isomer of fluoxetine is disclosed in U.S. Pat. Nos. 5,648,396 and 5,708,035, the contents of which are incorporated by reference.

Unless otherwise indicated herein, when the name of a chiral drug is used without modification that name refers to the racemic mixture, the (+) and (−) isomers and mixtures thereof. For example, “pindolol” refers to racemic pindolol, S(+) pindolol, R(−) pindolol and mixtures of S(+) and R(−) pindolol.

The term benzodiazepine refers to the portion of the structure composed of a benzene ring (A) fused to a seven-membered diazepine ring (B) (Goodman & Gilman, The Pharmacological Basis of Therapeutics, 9th Ed. McGraw-Hill, 1996, Chapter 17, p 363). However, since-all the important benzodiazepines contain a 5-aryl substituent (ring C) and a 1,4-diazepine ring, the term has come to mean the 5-aryl-1,4-benzodiazepines. Various modifications in the structure of the ring systems have yielded compounds with similar activities.

The effects of the benzodiazepines virtually all result from actions of these drugs on the CNS. The most prominent of these effects are sedation, hypnosis, decreased anxiety, muscle relaxation, anterograde amnesia, and anticonvulsant activity. A variety of benzodiazepine-like effects have been observed in vivo and in vitro and have been classified as full agonistic effects (i.e., faithfully mimicking agents such as diazepam with relatively low fractional occupancy of binding sites) or partial agonistic effects (i.e., producing less intense maximal effects and/or requiring relatively high fractional occupancy compared to agents such as diazepam).

Examples of benzodiazepines that can be used in the present invention include, but are not limited to, alprazolam, brotizolam, chlordiazepoxide, clobazam, clonazepam, clorazepate, demoxepam, diazepam, estazolam, flumazenil, flurazepam, halazepam, lorazepam, midazolam, nitrazepam, nordazepam, oxazepam, prazepam, quazepam, temazepam and triazolam (Goodman & Gilman, The Pharmacological Basis of Therapeutics, 9th Ed. McGraw-Hill, 1996, Chapter 17, p363, Table 17-1).

XANAX® tablets contain alprazolam which is a triazolo analog of the 1,4 benzodiazepine class of central nervous system-active compounds (Physician's Desk Reference® 52 Edition 1998, page 2294). The chemical name of alprazolam is 8-Chloro-1-methyl-6-phenyl-4H-s-triazolo[4,3-α][1,4]benzodiazepine. XANAX Tablets (alprazolam) are indicated for the management of anxiety disorder (a condition corresponding most closely to the APA Diagnostic and Statistical Manual [DSM-III-R] diagnosis of generalized anxiety disorder) or the short-term relief of symptoms of anxiety.

LIBRIUM® (chlordiazepoxide HCl) is 7-chloro-2-(methylamino)-5-phenyl-3H-1,4-benzodiazepine 4-oxide hydrochloride (Physician's Desk Reference® 52 Edition 1998, page 2522). Librium is indicated for the management of anxiety disorders or for the short-term relief of symptoms of anxiety, withdrawal symptoms of acute alcoholism, and preoperative apprehension and anxiety.

KLONOPIN® (clonazepam) is 5-(2-chlorophenyl)-1,3-dihydro-7-nitro-2H-1,4-benzodiazepin-2-one (Physician's Desk Reference® 52 Edition 1998, page 2475). Klonopin is useful alone or as an adjunct in the treatment of the Lennox-Gastaut syndrome (petit mal variant), akinetic and myoclonic seizures. Klonopin is also indicated for the treatment of panic disorder, with or without agoraphobia, as defined in DSM-IV.

TRANXENE® (clorazepate dipotassium) is indicated for the management of anxiety disorders or for the short-term relief of the symptoms of anxiety, as adjunctive therapy in the management of partial seizures, and for the symptomatic relief of acute alcohol withdrawal (Physician's Desk Reference® 52 Edition 1998, pages 472-473).

VALIUM® (diazepam) is a benzodiazepine derivative. Chemically, diazepam is 7-chloro-1,3-dihydro-1-methyl-5-phenyl-2H-1,4-benzodiazepin-2-one (Physician's Desk Reference® 52 Edition 1998, page 2527). Valium is indicated for the management of anxiety disorders or for the short-term relief of the symptoms of anxiety.

PROSOM™ (estazolam tablets) is 8-chloro-6-phenyl-4H-s-triazolo[4-3-α][1,4]benzodiazepine (Physician's Desk Reference® 52 Edition 1998, pages 470-471). ProSom (estazolam) is indicated for the short-term management of insomnia characterized by difficulty in falling asleep, frequent nocturnal awakenings, and/or early morning awakenings.

ROMAZICON® (flumazenil) is a benzodiazepine receptor antagonist (Physician's Desk Reference® 52 Edition 1998, pages 2494-2495). Chemically, flumazenil is ethyl 8-fluoro-5,6-dihydro-5-methyl-6-oxo-4H-imidazo(1,5-a](1,4)benzodiazepine-3-carboxylate. ROMAZICON is indicated for the complete or partial reversal of the sedative effects of benzodiazepines in cases where general anesthesia has been induced and/or maintained with benzodiazepines, where sedation has been produced with benzodiazepines for diagnostic and therapeutic procedures, and for the management of benzodiazepine overdose.

DALMANE® (flurazepam hydrochloride) is chemically 7-chloro-1-[2-(di-ethylamino)ethyl]-5-(o-fluorophenyl)-1,3-dihydro-2H-1,4-benzodiazzepin-2-one dihydrochloride (Physician's Desk Reference® 52 Edition 1998, page 2520). Dalmane is a hypnotic agent useful for the treatment of insomnia characterized by difficulty in falling asleep, frequent nocturnal awakenings, and/or early morning awakenings.

ATIVAN® (lorazepam) has the chemical formula, 7-chloro-5-(o-chlorophenyl)-1,3-dihydro-3-hydroxy-2H-1,4-benzodiazepin-2-one (Physician's Desk Reference® 52 Edition 1998, page 3013). Ativan (lorazepam) is indicated for the management of anxiety disorders or for the short-term relief of the symptoms of anxiety or anxiety associated with depressive symptoms.

VERSED® (midazolam HCl) is 8-chloro-6-(2-fluorophenyl)1-methyl-4H-imidazo[1,5-a](1,4]benzodiazepine (Physician's Desk Reference® 52 Edition 1998, pages 2512-2513).

SERAX® (oxazepam) is the first of a chemical series of compounds known as the 3-hydroxybenzodiazepinones (Physician's Desk Reference® 52 Edition 1998, page 3132). Serax (oxazepam) is indicated for the management of anxiety disorders or for the short-term relief of the symptoms of anxiety.

DORAL® (quazepam) is a trifluoroethyl benzodiazepine hypnotic agent, having the chemical name 7-chloro-5-(o-fluoro-phenyl)-1,3-dihydro-1-(2,2,2-trifluoroethyl)2H-1,4-benzodiazepine-2-thione (Physician's Desk Reference® 52 Edition 1998, page 2958). DORAL is indicated for the treatment of insomnia characterized by difficulty in falling asleep, frequent nocturnal awakenings, and/or early morning awakenings.

RESTORIL® (temazepam) is a benzodiazepine hypnotic agent. The chemical name is 7-chloro-1,3-dihydro-3-hydroxy-1-methyl-5-phenyl-2H-1,4-benzodiazepin-2-one (Physician's Desk Reference® 52 Edition 1998, page 1894). RESTORIL® (temazepam) is indicated for the short-term treatment of insomnia (generally 7-10 days).

HALCION® Tablets contain triazolam, a triazologenzodiazepine hypnotic agent (Physician's Desk Reference® 52 Edition 1998, pages 2275-2276). HALCION is indicated for the short-term treatment of insomnia (generally 7-10 days).

Antipsychotic agents are used primarily in the management of patients with psychotic or other serious psychiatric illness marked by agitation and impaired reasoning. These drugs have other properties that possibly are useful clinically, including antiemetic and antihistamine effects and the ability to potentiate analgesics, sedatives, and general anesthetics. Antipsychotic agents can also be used with optically pure R(−) fluoxetine. Tricyclic antipsychotic drugs fall into three subtypes: phenothiazines, thioxanthenes and other heterocyclic compounds.

Examples of phenothiazines include, but are limited to: THORAZINE® (chlorpromazine), SERENTIL® (mesoridazine besylate), MELLARIL® or MILLAZINE® (thioridazine), TINDAL® (acetophenazine), PERMITIL® or PROLIXIN® (fluphenazine) TRILAFON® (perphenazine) and STELAZINE® (trifluoperazine) (Goodman & Gilman, The Pharmacological Basis of Therapeutics, 9th Ed. McGraw-Hill, 1996, Chapter 18, p404, Table 18-1).

THORAZINE® (chlorpromazine) is 10-(3-dimethylaminopropyl)-2-chlorphenothiazine, a dimethylamine derivative of phenothiazine (Physician's Desk Reference® 52 Edition 1998, pages 2870-2871). THORAZINE® (chlorpromazine) is indicated, inter alia, for the management of manifestations of psychotic disorders.

SERENTIL® (mesoridazine besylate) is 10-[2(1-methyl-2-piperidyl)ethyl]-2-methyl-sylfinyl)-phenothiazine (Physician's Desk Reference® 52 Edition 1998, pages 725-726). Serentil® (mesoridazine besylate) is indicated in the treatment of, inter alia, emotional withdrawal, conceptual disorganization, anxiety, tension, hallucinatory behavior, suspiciousness and blunted affect in schizophrenic patients.

TRILAFON® (perphenazine) is (4-[3-(2-chlorophenothiazin-10-yl)propyl-1-piperazineethanol) (Physician's Desk Reference® 52 Edition 1998, page 2666). TRILAFON® (perphenazine) is indicated for use in the management of the manifestations of psychotic disorders and for the control of severe nausea and vomiting in adults.

STELAZINE® (trifluoperazine) is indicated for the management of the manifestations of psychotic disorders and for the short-term treatment of generalized non-psychotic anxiety (Physician's Desk Reference® 52 Edition 1998, page 2862).

Examples of thioxanthenes include, but are limited to: TARACTAN® (chlorprothixene) and NAVANE® (thiothixene) (Goodman & Gilman, The Pharmacological Basis of Therapeutics, 9th Ed. McGraw-Hill, 1996, Chapter 18, p404, Table 18-1).

NAVANE® (thiothixene) is the cis isomer of N,N-dimethyl-9-[3-(4-methyl-1-piperazinyl)-propylidene]thioxanthene-2-sulfonamide (Physician's Desk Reference® 52 Edition 1998, page 2192). NAVANE® (thiothixene) is indicated in the management of manifestations of psychotic disorders.

Examples of the other heterocyclic compounds that can be used in combination with R(−) fluoxetine include, but are limited to: CLOZARIL® (clozapine), HALDOL® (haloperidol), LOXITANE® (loxapine), MOBAN® (molindone), ORAP® (Pimozide), RISPERDAL® (risperidone), 9-hydroxy-risperidone and NORPRAMIN® (desipramine hydrochloride) (Goodman & Gilman, The Pharmacological Basis of Therapeutics, 9th Ed. McGraw-Hill, 1996, Chapter 18, p404, Table 18-1).

CLOZARIL® (clozapine) is a tricyclic dibenzodiazepine derivative, 8-chloro-11-(4-methyl-1-piperazinyl)5H-dibenzo[b,e][1,4]diazepine (Physician's Desk Reference® 52 Edition 1998, page 1834). CLOZARIL® (clozapine) is indicated for the management of severely ill schizophrenic patients who fail to respond adequately to standard antipsychotic drug treatment.

HALDOL® (haloperidol) is 4-[4-(p-chlorophenyl)-4-hydroxy-piperidonol-4′-fluorobutyrophenone (Physician's Desk Reference® 52 Edition 1998, pages 1997-1998). HALDOL® (haloperidol) is indicated, inter alia, for use in the management of manifestations of psychotic disorders, for the control of tics and vocal utterances of Tourette's Disorder in children and adults, for the treatment of severe behavior problems in children of combative.

LOXITANE® (loxapine) is 2-Chloro-11-(4-methyl-1-piperazinyl)dibenz[b,f][1-4]oxaxepine (Physician's Desk Reference® 52 Edition 1998, page 1396). LOXITANE® (loxapine) is indicated for the management of the manifestations of psychotic disorders.

MOBAN® (molindone) is 3-ethyl-6,7-dihydro-2-methyl-5(morpholinomethyl) indol-4(5H)-one hydrochloride (Physician's Desk Reference® 52 Edition 1998, pages 976-977). MOBAN® (molindone) is indicated for the management of the manifestations of psychotic disorders.

ORAP® (Pimozide) is 1-[1-[4,4-bis(4-fluorophenyl)butyl]4-piperidinyl]-1,3-dihydro-2H-benzimidazole-2-one (Physician's Desk Reference®52 Edition 1998, page 978). ORAP® (Pimozide) is indicated for the suppression of motor and phonic tics in patients with Tarried's Disorder who have failed to respond satisfactorily to standard treatment.

RISPERDAL® (risperidone) is 3-[2-[4-(6-fluoro-1,2-benzisoxazol-3-yl)-1-piperidinyl]ethyl]-6,7,8,9-tetrahydro-2-methyl-4H-pyrido[1,2-a]pyrimidin-4-one (Physician's Desk Reference® 52 Edition 1998, pages 1309-1310). RISPERDAL® (risperidone) is indicated for the management of the manifestations of psychotic disorders. 9-hydroxy-risperidone is a major active metabolite of risperidone (Id.). This major metabolite has been reported to be approximately equieffective as risperidone in Lacombe, Can. J. Psychiatry, 38 (Suppl.):S80-S88 (1993). 9-hydroxy-risperidone can be prepared from risperidone or from other well known starting materials using synthetic techniques well known in the art.

NORPRAMIN® (desipramine hydrochloride) is 5-H-Dibenz[bf]azepine-5-propanamine, 10, 11- dihydro-N-methyl-, monohydrochloride (Physician's Desk Reference® 52 Edition 1998, page 1227). NORPRAMIN® (desipramine hydrochloride) is indicated for the treatment of depression.

Examples of 5-HT_{1A} receptor antagonists and/or Badrenergic antagonists that can be used with R(−) fluoxetine in accordance with this invention include, but are limited to, alprenolol, WAY 100135, spiperone, VISKEN™ (pindolol), (S)-UH-301, LEVATOL™ (penbutolol), INDERAL™ (propranolol) and tertatolol.

Alprenolol (1-(1-methylethyl)amino-3-[2-(2-propenyl)phenoxy]-2-propanol) was disclosed by Brandstrom et al., U.S, Pat. No. 3,466,325, which shows its preparation.

WAY 100135 (N-(t-butyl)-3-[4-(2-methoxphenyl)piperazin-1-yl]-2-phenylpropanamide) was disclosed by Abou-Gharbia et al., U.S. Pat. No. 4,988,814, which disclosed that the compound has affinity for the 5-HT_{1A} receptor. Cliffe et al., J. Med. Chem., 36:1509-10 (1993) showed that the compound is a 5-HT_{1A} antagonist.

Spiperone (8-[4-(4-fluorophenyl)-4-oxobutyl]-1-phenyl-1,3,8-triazaspiro[4,5]decan-4-one) is a well-known compound, taught in U.S. Pat. 3,155,669 and 3,155,670. Its activity as a 5-HT_{1A} antagonist is shown by Middlmiss et al., Neurosci. and Biobehav. Rev., 16:75-82, (1992).

Pindolol (4-(2-hydroxy-3-isopropylaminopropoxy)-indole) was disclosed by Troxler et al., U.S. Pat. 3,471,515, which describes this compound as well as a beta-blocker. Dreshfield et al., Neurochem. Res., 21(5):557-562 (1996) disclosed that S(−) pindolol is a 5-HT_{1A} antagonist. VISKE™ (pindolol) is indicated in the management of hypertension (Physician's Desk Reference® 51 Edition 1997, page 2429). As used in the present application, the term “pindolol” refers to any acid addition salt or the free base, and includes either the racemic mixture or either of the R and S enantiomers.

(S)-UH-301 ((S)-5-fluoro-8-hydroxy-2-dipropylaminotetralin) is well known to pharmacologists and pharmaceutical chemists. Hillyer et al. taught its synthesis in J. Med. Chem., 33:1541-44 (1990) and Moreau et al., Brain Res. Bull., 29:901-04 (1992) provided considerable in vivo data about the compound.

Penbutolol (1-(t-butylamino)-2-hydroxy-3-(2-cyclopentylphenoxy)propane) was taught by Ruschig et al., U.S. Pat. No. 3,551,493, which describes it as a beta-blocker. Both the (−) and the (+) enantiomers of penbutolol are of interest; the (−) enantiomer is preferred for the present purpose but both enantiomers and the racemic mixture are included in the word “penbutolol” in this document. LEVATOL™ (penbutolol) is indicated in the management of hypertension (Physician's Desk Reference® 51 Edition 1997, page 2547).

Propranolol (1-isopropylamino-3-(1-naphthalenyloxy)-2propanol) was disclosed by Crowther et al., U.S. Pat. No. 3,337,628 to be a beta-blocker like tertatolol. INDERAL® (propranolol) is indicated in the management of hypertension. INDERAL® (propranolol) is also indicated in the management of myocardial infarction, migraine, essential tremor, hypertrophic subaortic stenosis and pheochromocytoma (Physician's Desk Reference® 51 Edition 1997, pages 2833-2834).

Tertatolol (8-(3-t-butylamino-2-hydroxypropyloxy)-thiochroman) was disclosed by Malen et al., U.S. Pat. No. 3,960,891, which teaches it to be a blocker of cardiac beta-adrenergic receptors. Its other activities, including the presently used 5-HT_{1A} antagonist activity, have been discovered since the original patents appeared.

Examples of non-benzodiazepine and non-tricyclic agents that can be used in this invention include, but are limited to: ZYPREXA™ (olanzapine), BUSPAR® (buspirone hydrochloride), ATARAX® (hydroxyzine hydrochloride), tomoxetine, SEROQUEL® (quetiapine), sertindole and ziprasidone.

ZYPREXA™ (olanzapine) is 2-methyl-4-(4-methyl-1-piperazinyl)-10H-thieno[2,3-b)[1,5]benzodiazepine (Physician's Desk Reference® 52 Edition 1998, page 1512). ZYPREXA™ (olanzapine) is indicated for the management of the manifestations of psychotic disorders.

BUSPAR® (buspirone hydrochloride) is an anti-anxiety agent that is not chemically or pharmacologically related to the benzodiazepines, barbiturates, or other sedative/anxiolytic drugs (Physician's Desk Reference® 52 Edition 1998, page 782). Chemically, buspirone hydrochloride is 8-[4-[4-(2-pyrimidinyl)-1-piperazinyl]butyl]-8-azaspiro(4,5)decane-7,9-dione monohydrochlordie. BUSPAR® (buspirone hydrochloride) is indicated for the treatment of Generalized Anxiety Disorder (ADA).

ATARAX® (hydroxyzine hydrochloride) is designated chemically as 1-(p-chlorobenzhydryl)- 4[2-(2-hydroxyethoxy)-ethyl] piperazine dihydrochloride (Physician's Desk Reference® 52 Edition 1998, page 2164). ATARAX® (hydroxyzine hydrochloride) is indicated for symptomatic relief of anxiety and tension associated with psychoneurosis, as an adjunct in organic disease states in which anxiety is manifested, in the management of puritus due to allergic conditions, and as sedative when used as premedication and following general anesthesia.

SEROQUEL® (Zeneca Pharmaceuticals) (quetiapine) has recently approved by the FDA to manage the manifestations of psychotic disorders (Medical Sciences Bulletin, Issue No. 241 (1997)). Although the exact mechanism of quetiapine is unknown, it is generally believed that the antipsychotic action of quetiapine is largely due to antagonism at the dopamine and serotonin receptors (Arvanitis, et al., Biol. Psychiatry, 15:233-246 (1997); Faustman et al., J. Clin. Psychopharmacol., 16:464-466 (1996); Fleischhacker et al., Drugs, 53:915-929 (1997); Small et al., Arch. Gen. Psychiatry, 54:549-557 (1997)).

Sertindole is designated chemically as 1-[2-[4-[5-Chloro-1-(4-fluorophenyl)-1H-indol-3-yl]-1-piperdinyl]ethyl]-2-imidazolidinone (The Merck Index, 12th Ed., Merck & Co., Inc., Whitehouse Station, N.J., 1996, page 1455). Sertindole can be prepared according to the procedures disclosed in U.S. Pat. No. 4,710,500, the content of which is incorporated herein by reference. Sertindole is a new atypical antipsychotic agent for the treatment of schizophrenia (Brown, et al., Pharmacotherapy, 18(1):69-83 (1998)).

Ziprasidone is designated chemically as 5-[2-[4-(1,2-Benzisothiazol-yl)-1-piperazinyl]ethyl]-6-chloro-1,3-dihydro-2H-indol-2-one (The Merck Index, 12th Ed., Merck & Co., Inc., Whitehouse Station, N.J., 1996, page 1737). Ziprasidone can be prepared according to the procedures disclosed in U.S. Pat. No. 4,831,031, the content of which is incorporated herein by reference. Ziprasidone is a novel antipsychotic agent that is in late clinical trials. It has shown antipsychotic efficacy in patients with schizophrenia and schizo-affective disorder and anxiolytic efficacy in patients about to undergo dental surgery (Davis and Markham, CNS Drugs, 8(2):153-159 (1997)).

While all combinations of R(−) fluoxetine and one or more above described biologically active compounds are useful and valuable, certain combinations are particularly valued and are preferred, as follows:

R(−) fluoxetine/alprazolam

R(−) fluoxetine/brotizolam

R(−) fluoxetine/chlordiazepoxide

R(−) fluoxetine/clobazam

R(−) fluoxetine/clonazepam

R(−) fluoxetine/clorazepate

R(−) fluoxetine/demoxepam

R(−) fluoxetine/diazepam

R(−) fluoxetine/estazolam

R(−) fluoxetine/flumazenil

R(−) fluoxetine/flurazepam

R(−) fluoxetine/halazepam

R(−) fluoxetine/lorazepam

R(−) fluoxetine/midazolam

R(−) fluoxetine/nitrazepam

R(−) fluoxetine/nordazepam

R(−) fluoxetine/oxazepam

R(−) fluoxetine/prazepam

R(−) fluoxetine/quazepam

R(−) fluoxetine/temazepam

R(−) fluoxetine/triazolam

R(−) fluoxetine/chlorpromazine

R(−) fluoxetine/mesoridazine

R(−) fluoxetine/thioridazine

R(−) fluoxetine/acetophenazine

R(−) fluoxetine/fluphenazine

R(−) fluoxetine/perphenazine

R(−) fluoxetine/trifluoperazine

R(−) fluoxetine/chlorprothixene

R(−) fluoxetine/thiothixene

R(−) fluoxetine/clozapine

R(−) fluoxetine/haloperidol

R(−) fluoxetine/lloxapine

R(−) fluoxetine/molindone

R(−) fluoxetine/pimozide

R(−) fluoxetine/risperidone

R(−) fluoxetine/9-hydroxy-risperidone

R(−) fluoxetine/alprenolol

R(−) fluoxetine/WAY 100135

R(−) fluoxetine/spiperone

R(−) fluoxetine/S(−) pindolol

R(−) fluoxetine/R(+) pindolol

R(−) fluoxetine/racemic pindolol

R(−) fluoxetine/(S)-UH-301

R(−) fluoxetine/penbutolol

R(−) fluoxetine/propranolol

R(−) fluoxetine/tertatolol

R(−) fluoxetine/desipramine

R(−) fluoxetine/clonidine

R(−) fluoxetine/olanzapine

R(−) fluoxetine/methylphenidate

R(−) fluoxetine/buspirone

R(−) fluoxetine/hydroxyzine

R(−) fluoxetine/tomoxetine

R(−) fluoxetine/quetiapine

R(−) fluoxetine/sertindole

R(−) fluoxetine/ziprasidone

The magnitude of a prophylactic or therapeutic dose of R(−) fluoxetine, of course, vary with the nature of the severity of the condition to be treated and its route of administration. It will also vary according to the age, weight and response of the individual patient. In general the daily dose range of R(−) fluoxetine in treatment of diseases or disorders, especially psychotic or psychiatric diseases or disorders lie within the range from about 1 mg to about 100 mg per day, preferably about 5 mg to about 60 mg per day, and most preferably from about 10 mg to about 40 mg per day, in single or divided doses. Effective amounts of the therapeutic agents to be used in combination with R(−) fluoxetine are based on the recommended doses known to those skilled in the art for the various diseases or disorders. It should be noted that the attending physician would know how to and when to terminate, interrupt, or adjust therapy to lower dosage due to toxicity, bone marrow, liver or kidney dysfunctions or adverse drug-drug interaction. Conversely, the attending physician would also know to adjust treatment to higher levels if the clinical response is not adequate (precluding toxicity).

A therapeutically effective dose refers to that amount of the compound sufficient to result in amelioration of symptoms or a prolongation of survival in a patient. As used herein, the term “an effective amount” and the term “a therapeutically effective dose/amount” has the same meaning. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. The data obtained from these cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 i.e., the concentration of the test compound which achieves a half-maximal inhibition of its target from infected cells compared to untreated control as determined in cell culture. Such information can be used to more accurately determine useful doses in humans.

In a certain embodiment of the invention, that is, a method of treating or preventing a psychotic or psychiatric disease or disorder, such as depression, an effective amount of R(−) fluoxetine and an effective amount of a biologically active 5-HT_{1A} receptor antagonist is administered for a first period of time; and then an effective amount of R(−) fluoxetine and a reduced amount of said biologically active 5-HT_{1A} receptor antagonist is administered for a second period of time. Preferably, 5-HT_{1A} receptor antagonist used herein is pindolol. Also preferably, the first period of time is from about 2 to about 6 weeks and the second period of time is from about 2 to about 20 days. A kit which comprises a plurality of dosage forms each comprising an effective amount of R(−) fluoxetinel a plurality of dosage forms each comprising an effective amount of a biologically active 5-HT_{1A} receptor antagonist, and a plurality of dosage forms each comprising a reduced amount of said biologically active 5-HT_{1A} receptor antagonist can be used in the above method. A kit which comprises a single unit dosage form of an effective amount of R(−) fluoxetine, a single unit dosage form of an effective amount of a biologically active 5-HT_{1A} receptor antagonist, and a single unit dosage form of a reduced amount of said biologically active 5-HT_{1A} receptor antagonist can also be used in the above method.

Any suitable route of administration may be employed for providing the patient with an effective dosage of the pharmaceutical compositions of the present invention. For example, oral, rectal, parenteral, transdermal, sublingual subcutaneous, intramuscular, inhalation and the like may be employed. Dosage forms include tablets, troches, dispersions, suspensions, solutions, capsules, patches and the like.

The pharmaceutical compositions of the present invention comprise R(−) fluoxetine and other active ingredients or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic acids including inorganic adds and organic acids.

Since R(−) fluoxetine, one of the key component of the pharmaceutical composition of the present invention, is basic, salts may be prepared from pharmaceutically acceptable nontoxic acids, including inorganic and organic acids. Such acids include acetic, benzenesulfonic, benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic and the like. Particularly preferred are hydrobromic, hydrochloric, phosphoric and sulfuric acids.

The compositions include compositions suitable for oral, rectal, parenteral (including subcutaneous, intramuscular, and intravenous), although the most suitable route in any given case will depend on the nature and severity of the condititm being treated. The most preferred route of the present invention is oral. They may be conveniently presented in unit dosage form and prepared by any of the methods well-known in the art of pharmacy.

The pharmaceutical carrier, which is included in the pharmaceutical compositions of the present invention, may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixir and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. The most preferred solid oral preparation is capsules. Because of their ease of administration, tablets and capsules represent the most advantageous oral dosage unit form, in which case solid pharmaceutical carriers are obviously employed. If desired, tablets may be coated by standard aqueous or nonaqueous techniques.

In addition to the common dosage forms set out above, the pharmaceutical compositions of the present invention may also be administered by controlled release means and/or delivery devices such as those described in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533 and 5,733,566, the disclosures of which are hereby incorporated by reference. The use of a racemic mixture of fluoxetine in a sustained release formulation is disclosed and/or claimed in U.S. Pat. Nos. 4,797,286 and 4,947,092.

Pharmaceutical compositions of the present invention suitable for oral administration may be presented as discrete units such as capsules, cachets or tablets each containing a predetermined amount of the active ingredient, as a powder orgranules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Various references are cited herein, the disclosures of which are incorporated by reference in their entireties.

What is claimed is:

1. A pharmaceutical composition which comprises an effective amount of R(−) fluoxetine or a pharmaceutically acceptable salt thereof, substantially free of its S(+) stereoisomer, an effective amount of a biologically active benzodiazepine and a pharmaceutically acceptable carrier.

2. The pharmaceutical composition of claim 1, wherein said biologically active benzodiazepine is selected from the group consisting of alprazolam, brotizolam, chlordiazepoxide, clobazam, clonazepam, clorazepate, demoxepam, diazepam, estazolam, flumazenil, flurazepam, halazepam, lorazepam, midazolam, nitrazepam, nordazepam, oxazepam prazepam, quazepam, temazepam and triazolam.

3. The pharmaceutical composition of claim 1, wherein the amount of R(−) fluoxetine is from about 5 mg to about 100 mg.

4. The pharmaceutical composition of claim 1, wherein the R(−) fluoxetine is present in an amount on weight basis, relative to S(+) fluoxetine, from about 100:1 to about 10,000:1.

5. The pharmaceutical composition of claim 1, wherein the pharmaceutically acceptable salt of R(−) fluoxetine is R(−) fluoxetine hydrochloride.

6. A pharmaceutical composition which comprises an effective amount of R(−) fluoxetine or a pharmaceutically acceptable salt thereof, substantially free of its S(+) stereoisomer, an effective amount of a biologically active tricyclic antipsychotic compound and a pharmaceutically acceptable carrier.

7. The pharmaceutical composition of claim 6, wherein said biologically active tricyclic antipsychotic compound is selected from the group consisting of chlorpromazine, mesoridazine, thioridazine, acetophenazine, fluphenazine, perphenazine, trifluoperazine, chlorprothixene, thiothixene, clozapine, haloperidol, loxapine, molindone, pimozide, risperidone, 9-hydroxy-risperidone and desipramine.

8. The pharmaceutical composition of claim 6, wherein the amount of the R(−) fluoxetine is from about 5 mg to about 100 mg.

9. The pharmaceutical composition of claim 6, wherein the R(−) fluoxetine is present in an amount on weight basis, relative to S(+) fluoxetine, from about 100:1 to about 10,000:1.

10. The pharmaceutical composition of claim 6, wherein the pharmaceutically acceptable salt of R(−) fluoxetine is R(−) fluoxetine hydrochloride.

11. A pharmaceutical composition which comprises an effective amount of R(−) fluoxetine or a pharmaceutically acceptable salt thereof, substantially free of its S(+) stereoisomer, an effective amount of a biologically active 5-HT_{1A} receptor antagonist and a pharmaceutically acceptable carrier.

12. The pharmaceutical composition of claim 11, wherein said biologically active 5-HT_{1A} receptor antagonist is selected from the group consisting of alprenolol, WAY 100135, spiperone, pindolol, (S)-UH-301, penbutolol, propranolol and tertatolol.

13. The pharmaceutical composition of claim 11, wherein the amount of R(−) fluoxetine is from about 5 mg to about 100 mg.

14. The pharmaceutical composition of claim 11, wherein the R(−) fluoxetine is present in an amount on weight basis, relative to S(+) fluoxetine, from about 100:1 to about 10,000:1.

15. The pharmaceutical composition of claim 11, wherein the pharmaceutically acceptable salt of R(−) fluoxetine is R(−) fluoxetine hydrochloride.

16. A pharmaceutical composition which comprises an effective amount of R(−) fluoxetine or a pharmaceutically acceptable salt thereof, substantially free of its S(+) stereoisomer, an effective amount of a biologically active compound selected from the group consisting of buspirone, hydroxyzine, lorazepam, olanzapine, quetiapine, risperidone, 9-hydroxy-risperidone, sertindole, tomoxetine, valium and ziprasidone and a pharmaceutically acceptable carrier.

17. The pharmaceutical composition of claim 16, wherein the amount of R(−) fluoxetine is from about 5 mg to about 100 mg.

18. The pharmaceutical composition of claim 16, wherein the R(−) fluoxetine is present in an amount on weight basis, relative to S(+) fluoxetine, from about 100:1 to about 10,000:1.

19. The pharmaceutical composition of claim 16, wherein the pharmaceutically acceptable salt of R(−) fluoxetine is R(−) fluoxetine hydrochloride.

20. A pharmaceutical composition which comprises an effective amount of R(−) fluoxetine or a pharmaceutically acceptable salt thereof, substantially free of its S(+) stereoisomer, an effective amount of a biologically active compound selected from the group consisting of racemic pindolol, S(−) pindolol and R(+) pindolol and a pharmaceutically acceptable carrier.

21. The pharmaceutical composition of claim 20, wherein the amount of R(−) fluoxetine is from about 5 mg to about 100 mg.

22. The pharmaceutical composition of claim 20, wherein the R(−) fluoxetine is present in an amount on weight basis, relative to S(+) fluoxetine, from about 100:1 to about 10,000:1.

23. The pharmaceutical composition of claim 20, wherein the pharmaceutically acceptable salt of R(−) fluoxetine is R(−) fluoxetine hydrochloride.

24. A pharmaceutical composition which comprises an effective amount of R(−) fluoxetine or a pharmaceutically acceptable salt thereof, substantially free of its S(+) stereoisomer, an effective amount of a biologically active 5-HT₃ receptor agonist, and a pharmaceutically acceptable carrier.

25. The pharmaceutical composition of claim 24, wherein the R(−) fluoxetine is present in an amount on weight basis, relative to S(+) fluoxetine, from about 100:1 to about 10,000:1.

26. The pharmaceutical composition of claim 24, wherein the pharmaceutically acceptable salt of R(−) fluoxetine is R(−) fluoxetine hydrochloride.

27. A method of treating or preventing a psychotic or psychiatric disease or disorder in a mammal which comprises administering to said mammal, to which such treatment or prevention is needed, an effective amount of the pharmaceutical composition of claim 1.

28. A method of treating or preventing a psychotic or psychiatric disease or disorder in a mammal which comprises administering to said-mammal, to which such treatment or prevention is needed, an effective amount of the pharmaceutical composition of claim 6.

29. A method of treating or preventing a psychotic or psychiatric disease or disorder in a mammal which comprises administering to said mammal, to which such treatment or prevention is needed, an effective amount of the pharmaceutical composition of claim 11.

30. A method of treating or preventing a psychotic or psychiatric disease or disorder in a mammal which comprises administering to said mammal, to which such treatment or prevention is needed, an effective amount of the pharmaceutical composition of claim 16.

31. A method of treating or preventing a psychotic or psychiatric disease or disorder in a mammal which comprises administering to said mammal, to which such treatment or prevention is needed, an effective amount of the pharmaceutical composition of claim 20.

32. A method of treating or preventing a psychotic or psychiatric disease or disorder in a mammal which comprises administering to said mammal, to which such treatment or prevention is needed, an effective amount of the pharmaceutical composition of claim 24.

33. A method of treating or preventing a disease or disorder in a mammal which comprises administering to said mammal, to which such treatment or prevention is needed, an effective amount of R(−) fluoxetine or a pharmaceutically acceptable salt thereof substantially free of its S(+) stereoisomer, and an effective amount of a biologically active benzodiazepine.

34. The method of claim 33, wherein the psychotic or psychiatric disease or disorder is depression.

35. The method of claim 33, wherein said biologically active benzodiazepine is selected from the group consisting of alprazolam, brotizolam, chlordiazepoxide, clobazam, clonazepam, clorazepate, demoxepam, diazepam, estazolam, flumazenil, flurazepam, halazepam, lorazepam, midazolam, nitrazepam, nordazepam, oxazepam, prazepam, quazepam, temazepam and triazolam.

36. The method of claim 33, wherein the amount of R(−) fluoxetine is from about 5 mg to about 100 mg.

37. The method of claim 33, wherein the R(−) fluoxetine is present in an amount on weight basis, relative to S(+) fluoxetine, from about 100:1 to about 10,000:1.

38. The method of claim 33, wherein the pharmaceutically acceptable salt of R(−) fluoxetine is R(−) fluoxetine hydrochloride.

39. The method of claim 33, wherein said disease or disorder is a psychotic or psychiatric disease or disorder.

40. The method of claim 39, wherein the disease or disorder is selected from the group consisting of anxiety disorder, depression, alcohol withdrawal, preoperative apprehension, Lennox-Gastaut syndrome (petit mal variant), seizures, panic disorder, skeletal muscle spasm, spasticity caused by upper motor neuron disorders, athetosis and stiffman syndrome, convulsive disorders and insomnia.

41. A method of treating or preventing a psychotic or psychiatric disease or disorder in a mammal which comprises administering to said-mammal, to which such treatment or prevention is needed, an effective amount of R(−) fluoxetine or a pharmaceutically acceptable salt thereof, substantially free of its S(+) stereoisomer, and an effective amount of a biologically active tricyclic antipsychotic compound.

42. The method of claim 41, wherein the psychotic or psychiatric disease or disorder is depression.

43. The method of claim 41, wherein said biologically active tricyclic antipsychotic compound is selected from the group consisting of chlorpromazine, mesoridazine, thioridazine, acetophenazine, fluphenazine, perphenazine, trifluoperazine, chlorprothixene, thiothixene, clozapine, haloperidol, loxapine, molindone, pimozide, risperidone, 9-hydroxy-risperidone and desipramine.

44. The method of claim 41, wherein the amount of the R(−) fluoxetine is from about 5 mg to about 100 mg.

45. The method of claim 41, wherein the R(−) fluoxetine is present in an amount on weight basis, relative to S(+) fluoxetine, from about 100:1 to about 10,000:1.

46. The method of claim 41, wherein the pharmaceutically acceptable salt of R(−) fluoxetine is R(−) fluoxetine hydrochloride.

47. The method of claim 43,,wherein the disease or disorder is selected from the group consisting of anxiety disorder, depression, nausea, vomiting, restlessness and apprehension before surgery, acute intermittent porphyria, tetanus, intractable hiccups, severe behavioral problems and hyperactivity in children, emotional withdrawal, conceptual disorganization, tension, hallucinatory behavior, suspiciousness and blunted affect in schizophrenic patients and Tourette's Disorder.

48. A method of treating or preventing a psychotic or psychiatric disease or disorder in a mammal which comprises administering to said mammal, to which such treatment or prevention is needed, an effective amount of R(−) fluoxetine, or a pharmaceutically acceptable salt thereof, substantially free of its S(+) stereoisomer, and an effective amount of a 5-HT_{1A} receptor antagonist.

49. The method of claim 48, wherein the psychotic or psychiatric disease or disorder is depression.

50. The method of claim 48, wherein the amount of R(−) fluoxetine is from about 5 mg to about 100 mg.

51. The method of claim 48, wherein the R(−) fluoxetine is present in an amount on weight basis, relative to S(+) fluoxetine, from about 100:1 to about 10,000:1.

52. The method of claim 48, wherein the pharmaceutically acceptable salt of R(−) fluoxetine is R(−) fluoxetine hydrochloride.

53. A method of treating or preventing a psychotic or psychiatric disease or disorder in a mammal which comprises administering to said mammal, to which such treatment or prevention is needed, an effective amount of R(−) fluoxetine, or a pharmaceutically acceptable salt thereof, substantially free of its S(+) stereoisomer, and an effective amount of a 5-HT₃ receptor agonist.

54. The method of claim 53, wherein the psychotic or psychiatric disease or disorder is depression.

55. The method of claim 53, wherein the amount of R(−) fluoxetine is from about 5 mg to about 100 mg.

56. The method of claim 53, wherein the R(−) fluoxetine is present in an amount on weight basis, relative to S(+) fluoxetine, from about 100:1 to about 10,000:1.

57. The method of claim 53, wherein the pharmaceutically acceptable salt of R(−) fluoxetine is R(−) fluoxetine hydrochloride.

58. A method of treating or preventing post-myocardial infarction depression in a mammal which comprises administering to said mammal, to which such treatment or prevention is needed, an effective amount of R(−) fluoxetine, or a pharmaceutically acceptable salt thereof, substantially free of its S(+) stereoisomer, and an effective amount of a β-adrenergic antagonist.

59. The method of claim 58, wherein the β-adrenergic antagonist is S(−) pindolol.

60. The method of claim 58, wherein the amount of R(−) fluoxetine is from about 5 mg to about 100 mg.

61. The method of claim 58, wherein the R(−) fluoxetine is present in an amount on weight basis, relative to S(+) fluoxetine, from about 100:1 to about 10,000:1.

62. The method of claim 58, wherein the pharmaceutically acceptable salt of R(−) fluoxetine is R(−) fluoxetine hydrochloride.

63. A method of treating or preventing posttraumatic stress disorder in a mammal which comprises administering to said mammal, to which such treatment or prevention is needed, an effective amount of R(−) fluoxetine, or a pharmaceutically acceptable salt thereof, substantially free of its S(+) stereoisomer, and an effective amount of a 5-HT_{1A} receptor antagonist.

64. The method of claim 63, wherein the 5-HT_{1A} receptor antagonist is S(−) pindolol or R(+) pindolol.

65. The method of claim 63, wherein the amount of R(−) fluoxetine is from about 5 mg to about 100 mg.

66. The method of claim 63, wherein the R(−) fluoxetine is present in an amount on weight basis, relative to S(+) fluoxetine, from about 100:1 to about 10,000:1.

67. The method of claim 63, wherein the pharmaceutically acceptable salt of R(−) fluoxetine is R(−) fluoxetine hydrochloride.

68. A method of treating or preventing a psychotic or psychiatric disease or disorder in a mammal which comprises:
a) administering to said mammal an effective amount of R(−) fluoxetine or a pharmaceutically acceptable salt thereof substantially free of its S(+) stereoisomer, and an effective amount of a biologically active 5-HT_{1A} receptor antagonist for a first period of time; and
b) after the first period of time, administering to said mammal an effective amount of R(−) fluoxetine or a pharmaceutically acceptable salt thereof substantially free of its S(+) stereoisomer and a reduced amount of said biologically active 5-HT_{1A} receptor antagonist for a second period of time.

69. The method of claim 68, wherein the psychotic or psychiatric disease or disorder is depression.

70. The method of claim 68, wherein the biologically active 5-HT_{1A} receptor antagonist is S(−) pindolol or R(+) pindolol.

71. The method of claim 68, wherein the amount of R(−) fluoxetine is from about 5 mg to about 100 mg.

72. The method of claim 68, wherein the R(−) fluoxetine is present in an amount on weight basis, relative to S(+) fluoxetine, from about 100:1 to about 10,000:1.

73. The method of claim 68, wherein the pharmaceutically acceptable salt of R(−) fluoxetine is R(−) fluoxetine hydrochloride.

74. The method of claim 68, wherein the first period of time is about 2 to 6 weeks.

75. The method of claim 68, wherein the second period of time is about 5 to 20 days.

76. A kit which comprises:
a) a plurality of dosage forms each comprising an effective amount of R(−) fluoxetine or a pharmaceutically acceptable salt thereof substantially free of its S(+) stereoisomer;
b) a plurality of dosage forms each comprising an effective amount of a biologically active 5-HT_{1A} receptor antagonist; and
c) a plurality of dosage forms each comprising a reduced amount of said biologically active 5-HT_{1A} receptor antagonist.

77. The kit of claim 76, wherein the biologically active 5-HT_{1A} receptor antagonist is S(−) pindolol or R(+) pindolol.

78. The kit of claim 76, wherein the pharmaceutically acceptable salt of R(−) fluoxetine is R(−) fluoxetine hydrochloride.

79. A method of treating posttraumatic stress disorder in a human which comprises administering to said human, to which such treatment or prevention is needed, an effective amount of R(−) fluoxetine, or a pharmaceutically acceptable salt thereof, substantially free of its S(+) stereoisomer, and an effective amount of a biologically active compound.

80. A method of treating or preventing a disease or disorder in a mammal which comprises administering to said mammal, to which such treatment or prevention is needed, an effective amount of R(−) fluoxetine or a pharmaceutically acceptable salt thereof substantially free of its S(+) stereoisomer, and an effective amount of a compound selected from the group consisting of quetiapine, sertindole and ziprasidone.

81. The method of claim 80, wherein the disease or disorder is psychotic disease or disorder.

82. The method of claim 81, wherein the psychotic disease or disorder is schizophrenia.

83. The method of claim 80, wherein the amount of R(−) fluoxetine is from about 5 mg to about 100 mg.

84. The method of claim 80, wherein the R(−) fluoxetine is present in an amount on weight basis, relative to S(+) fluoxetine, from about 100:1 to about 10,000:1.

85. The method of claim 80, wherein the pharmaceutically acceptable salt of R(−) fluoxetine is R(−) fluoxetine hydrochloride.
